(19) United States
(12) Patent Application Publication
(10) Pub. No.: US 2026/0334907 A1
(43) Pub. Date: Oct. 29, 2026

(54) Medical X-Ray Imaging Method and Medical X-Ray Imaging Device

(71) Applicant: Siemens Shanghai Medical Equipment Ltd., Shanghai (CN)

(72) Inventors: Yan Shen, Shanghai (CN); Jun Kong, Shanghai (CN)

(73) Assignee: Siemens Shanghai Medical Equipment Ltd., Shanghai (CN)

(21) Appl. No.: 19/491,091

(22) PCT Filed: Jun. 3, 2024
(86) PCT No.: PCT/CN2024/097114

§ 371 Date: Dec. 8, 2025

(30) Foreign Application Priority Data
Jun. 12, 2023 (CN) 202310686059.X

(51) Int. Cl. A61B 6/00; A61B 6/04

(52) U.S. Cl. CPC A61B 6/545; A61B 6/0407; A61B 6/488; A61B 6/5241

(57) ABSTRACT

A medical X-ray imaging method is provided, including: setting an imaging target region; segmenting the imaging target region into a plurality of segmented regions according to first data, where the first data is a distribution of X-ray absorptivities of body parts of an examinee corresponding to the imaging target region; for each of the segmented regions, generating an X-ray photographing parameter for the segmented region according to an X-ray average absorptivity of a body part of the examinee corresponding to the segmented region and according to a correspondence between X-ray average absorptivities and X-ray photographing parameters; photographing a local X-ray image according to the X-ray photographing parameter corresponding to each of the segmented regions; and splicing the local X-ray images corresponding to the plurality of segmented regions to form a panoramic X-ray image of the imaging target region.

### TECHNICAL FIELD

The present disclosure relates to a medical imaging method, and in particular, to a medical X-ray imaging method and a medical X-ray imaging device for implementing the method.

### BACKGROUND

In clinical diagnosis, it is usually necessary to make a panoramic X-ray image to observe a complete bone structure, such as a complete spine or lower limb, of an examinee. However, existing X-ray imaging devices can only obtain X-ray images of local parts (namely, local X-ray images) of the complete bone structure in one shot, and then splice a plurality of local X-ray images to form a panoramic X-ray image. The complete bone structure of the examinee is dispersed in the plurality of local X-ray images. Currently, local X-ray images of the same panoramic X-ray image have the same size, and the image size is set according to a range of the panoramic X-ray image and a size of an overlapped part of adjacent local X-ray images. Since one local X-ray image has only one radiation dose, the same local X-ray image may have one part overexposed and the other part underexposed. For example, during the making of a panoramic X-ray image of a complete spine, the chest and the abdomen are located in the same local X-ray image, and there may be a case in which the chest is overexposed, and the abdomen is underexposed, causing poor uniformity of the finally formed panoramic X-ray image.

### SUMMARY

An object of the present disclosure is to provide a medical X-ray imaging method that is conducive to improving the uniformity of a panoramic X-ray image.

Another object of the present disclosure is to provide a medical X-ray imaging device, which is conducive to improving uniformity of a panoramic X-ray image.

The present disclosure provides a medical X-ray imaging method, including: S10: setting an imaging target region; S20: segmenting the imaging target region into a plurality of segmented regions according to first data, where the first data is a distribution of X-ray absorptivities of body parts of an examinee corresponding to the imaging target region, and for each of the segmented regions, a difference between a maximum value and a minimum value of an X-ray absorptivity of a body part of the examinee corresponding to the segmented region is not beyond a first preset difference range; S30: for each of the segmented regions, generating an X-ray photographing parameter for the segmented region according to an X-ray average absorptivity of the body part of the examinee corresponding to the segmented region and according to a correspondence between X-ray average absorptivities and X-ray photographing parameters; S40: photographing a local X-ray image according to the X-ray photographing parameter corresponding to each of the segmented regions; and S50: splicing the local X-ray images corresponding to the plurality of segmented regions to form a panoramic X-ray image of the imaging target region.

In the medical X-ray imaging method, the imaging target region is segmented into the plurality of segmented regions according to the distribution of the X-ray absorptivities of the body parts corresponding to the imaging target region of the examinee, and the X-ray photographing parameter for each of the segmented regions is generated according to the X-ray average absorptivity of the body part corresponding to the segmented region of the examinee and the correspondence between the X-ray average absorptivities and the X-ray photographing parameters. Since the X-ray absorptivities in the body parts corresponding to the same segmented region are close, a consistent exposure degree of parts in the local X-ray images can be achieved, thereby improving the uniformity of the panoramic X-ray image.

In another exemplary implementation of the medical X-ray imaging method, the distribution of the X-ray absorptivities of the body parts of the examinee corresponding to the imaging target region is represented as positions and sizes of local parts of the examinee, where the local parts of the examinee do not overlap with each other, and distinction of the local parts is related to the distribution of the X-ray absorptivities of the examinee. In step S20, each of the segmented regions covers only a part or all of one local part of the examinee. In this way, it is conducive to reducing an amount of computation and improving efficiency.

In still another exemplary implementation of the medical X-ray imaging method, one of the local parts is the head, neck, head and neck, chest, abdomen, upper limb, or lower limb.

In yet another exemplary implementation of the medical X-ray imaging method, step S20 includes: obtaining the first data according to an optical image of the examinee. The optical image is a visible light image or a pre-scanned X-ray image. The optical image is a 2D image or a 3D image. In this way, the first data can be obtained conveniently.

In yet another exemplary implementation of the medical X-ray imaging method, the optical image is a head image of the examinee. Step S20 includes: obtaining a position and a size of the head of the examinee through measurement according to the head image of the examinee, estimating positions and sizes of body parts of the examinee according to the position and the size of the head of the examinee, and obtaining the first data according to the positions and the sizes of the body parts of the examinee. In this way, the first data can be obtained conveniently.

In yet another exemplary implementation of the medical X-ray imaging method, the optical image is a whole body image of the examinee. Step S20 includes: obtaining positions and sizes of body parts of the examinee according to the whole body image of the examinee, and obtaining the first data according to the positions and the sizes of the body parts of the examinee. In this way, the first data can be obtained conveniently.

In yet another exemplary implementation of the medical X-ray imaging method, the examinee is borne on a bearing surface of a medical bed. The bearing surface has a bearing region. A plurality of pressure detection portions are arranged in the bearing region. The medical bed can detect a pressure of the examinee on the medical bed in a gravity direction at each of the pressure detection portions. Step S20 includes: estimating positions and sizes of body parts of the examinee according to the pressure of the examinee on the medical bed in the gravity direction at the pressure detection portions, and obtaining the first data according to the positions and the sizes of the body parts of the examinee. In this way, the first data can be obtained conveniently.

In yet another exemplary implementation of the medical X-ray imaging method, step S20 includes: estimating positions and sizes of body parts of the examinee according to a height, a weight, and a body fat ratio of the examinee, and obtaining the first data according to the positions and the sizes of the body parts of the examinee. In this way, the first data can be obtained conveniently.

In yet another exemplary implementation of the medical X-ray imaging method, step S30 includes: for each of the segmented regions, generating an X-ray photographing parameter for the segmented region according to the local part of the examinee corresponding to the segmented region and according to a correspondence between the local parts and X-ray photographing parameters. In this way, it is conducive to improving the uniformity of the panoramic X-ray image.

In yet another exemplary implementation of the medical X-ray imaging method, the correspondence between the local parts and X-ray photographing parameters is determined according to an X-ray photographing parameter corresponding to a case in which an average gray scale of historical X-ray images of the local parts at the same photographing angle meets a first preset gray scale range and/or a contrast between two same organs meets a first preset contrast range. In this way, it is conducive to improving the uniformity of the panoramic X-ray image.

In yet another exemplary implementation of the medical X-ray imaging method, edges of photographed regions corresponding to adjacent segmented regions overlap with each other. In step S30, for each of the segmented regions, the X-ray photographing parameter for the segmented region is further generated according to an X-ray photographing parameter corresponding to a case in which an average gray scale of historical X-ray images of a body part corresponding to an overlapped part of the photographed regions corresponding to the segmented region meets a second preset gray scale range and/or a contrast between two same organs meets a second preset contrast range. In this way, it is conducive to reducing jumps of the gray scale and/or the contrast of the overlapped part.

In yet another exemplary implementation of the medical X-ray imaging method, the correspondence between X-ray average absorptivities and X-ray photographing parameters is determined according to an X-ray photographing parameter corresponding to a case in which an average gray scale of historical X-ray images of a body part meeting the X-ray average absorptivity at the same photographing angle meets a first preset gray scale range and/or a contrast between two same organs meets a first preset contrast range. In this way, it is conducive to improving the uniformity of the panoramic X-ray image.

In yet another exemplary implementation of the medical X-ray imaging method, edges of photographed regions corresponding to adjacent segmented regions overlap with each other. In step S30, for each of the segmented regions, the X-ray photographing parameter for the segmented region is further generated according to an X-ray photographing parameter corresponding to a case in which an average gray scale of historical X-ray images of a body part meeting an X-ray average absorptivity that is of a body part corresponding to an overlapped part of the photographed regions corresponding to the segmented region at the same photographing angle meets a second preset gray scale range and/or a contrast between two same organs meets a second preset contrast range. In this way, it is conducive to reducing jumps of the gray scale and/or the contrast of the overlapped part.

In yet another exemplary implementation of the medical X-ray imaging method, the X-ray photographing parameters include a tube voltage, a tube current, an exposure time, and a photographed region.

In yet another exemplary implementation of the medical X-ray imaging method, the imaging target region is a rectangle, and the plurality of segmented regions are arranged in a direction parallel to a set of opposite edges of the rectangle.

The present disclosure further provides a medical X-ray imaging device, including a target region setting module, a segmentation module, a parameter generation module, a photographing module, and a splicing module. The target region setting module can set an imaging target region according to data input by a user. The segmentation module can segment the imaging target region into a plurality of segmented regions according to the first data. The first data is a distribution of X-ray absorptivities of body parts of an examinee corresponding to the imaging target region. The segmentation module can, for each of the segmented regions, maintain a difference between a maximum value and a minimum value of an X-ray absorptivity of a body part of the examinee corresponding to the segmented region, not beyond a first preset difference range. The parameter generation module can, for each of the segmented regions, generate an X-ray photographing parameter for the segmented region according to an X-ray average absorptivity of the body part of the examinee corresponding to the segmented region and according to a correspondence between X-ray average absorptivities and X-ray photographing parameters. The photographing module can photograph a local X-ray image according to the X-ray photographing parameter corresponding to each of the segmented regions. The splicing module can splice the local X-ray images corresponding to the plurality of segmented regions to form a panoramic X-ray image of the imaging target region.

In the medical X-ray imaging device, the imaging target region is segmented into the plurality of segmented regions according to the distribution of the X-ray absorptivities of the body parts corresponding to the imaging target region of the examinee, and the X-ray photographing parameter for each of the segmented regions is generated according to the X-ray average absorptivity of the body part corresponding to the segmented region of the examinee and the correspondence between the X-ray average absorptivities and the X-ray photographing parameters. Since the X-ray absorptivities in the body parts corresponding to the same segmented region are close, a consistent exposure degree of parts in the local X-ray images can be achieved, thereby improving the uniformity of the panoramic X-ray image.

In another exemplary implementation of the medical X-ray imaging device, the distribution of the X-ray absorptivities of the body parts of the examinee corresponding to the imaging target region is represented as positions and sizes of local parts of the examinee, where the local parts of the examinee do not overlap with each other, and distinction of the local parts is related to the distribution of the X-ray absorptivities of the examinee. The segmentation module can enable each of the segmented regions to cover only a part or all of one local part of the examinee. In this way, it is conducive to reducing an amount of computation and improving efficiency.

In still another exemplary implementation of the medical X-ray imaging device, one of the local parts is the head, neck, head and neck, chest, abdomen, upper limb, or lower limb.

In yet another exemplary implementation of the medical X-ray imaging device, the medical X-ray imaging device further includes a first data generation module. The first data generation module can obtain the first data according to an optical image of the examinee. The optical image is a visible light image or a pre-scanned X-ray image. In a case in which the optical image is a visible light image, the medical X-ray imaging device further includes a visible light image obtaining device. The visible light image obtaining device is configured to obtain the visible light image of the examinee. The optical image is a 2D image or a 3D image. In this way, the first data can be obtained conveniently.

In yet another exemplary implementation of the medical X-ray imaging device, the optical image is a head image of the examinee. The first data generation module can obtain a position and a size of the head of the examinee through measurement according to the head image of the examinee, estimate positions and sizes of body parts of the examinee according to the position and the size of the head of the examinee, and obtain the first data according to the positions and the sizes of the body parts of the examinee. In this way, the first data can be obtained conveniently.

In yet another exemplary implementation of the medical X-ray imaging device, the optical image is a whole body image of the examinee. The first data generation module can obtain positions and sizes of body parts of the examinee according to the whole body image of the examinee, and obtain the first data according to the positions and the sizes of the body parts of the examinee. In this way, the first data can be obtained conveniently.

In yet another exemplary implementation of the medical X-ray imaging device, the medical X-ray imaging device further includes a first data generation module. The examinee is borne on a bearing surface of a medical bed of the medical X-ray imaging device. The bearing surface has a bearing region. A plurality of pressure detection portions are arranged in the bearing region. The medical bed can detect a pressure of the examinee on the medical bed in a gravity direction at each of the pressure detection portions. The first data generation module can estimate positions and sizes of body parts of the examinee according to the pressure of the examinee on the medical bed in the gravity direction at the pressure detection portions, and obtain the first data according to the positions and the sizes of the body parts of the examinee. In this way, the first data can be obtained conveniently.

In yet another exemplary implementation of the medical X-ray imaging device, the medical X-ray imaging device further includes a first data generation module. The first data generation module can estimate positions and sizes of body parts of the examinee according to a height, a weight, and a body fat ratio of the examinee, and obtain the first data according to the positions and the sizes of the body parts of the examinee. In this way, the first data can be obtained conveniently.

In yet another exemplary implementation of the medical X-ray imaging device, the parameter generation module can, for each of the segmented regions, generate an X-ray photographing parameter for the segmented region according to the local part of the examinee corresponding to the segmented region and according to a correspondence between the local parts and X-ray photographing parameters. In this way, it is conducive to improving the uniformity of the panoramic X-ray image.

In yet another exemplary implementation of the medical X-ray imaging device, the medical X-ray imaging device further includes a correspondence generation module. The correspondence generation module can determine the correspondence between the local parts and X-ray photographing parameters according to an X-ray photographing parameter corresponding to a case in which an average gray scale of historical X-ray images of the local parts at the same photographing angle meets a first preset gray scale range and/or a contrast between two same organs meets a first preset contrast range. In this way, it is conducive to improving the uniformity of the panoramic X-ray image.

In yet another exemplary implementation of the medical X-ray imaging device, edges of photographed regions corresponding to adjacent segmented regions overlap with each other. The parameter generation module can, for each of the segmented regions, further generate the X-ray photographing parameter for the segmented region according to an X-ray photographing parameter corresponding to a case in which an average gray scale of historical X-ray images of a body part corresponding to an overlapped part of the photographed regions corresponding to the segmented region meets a second preset gray scale range and/or a contrast between two same organs meets a second preset contrast range. In this way, it is conducive to reducing jumps of the gray scale and/or the contrast of the overlapped part.

In yet another exemplary implementation of the medical X-ray imaging device, the medical X-ray imaging device further includes a correspondence generation module. The correspondence generation module can determine the correspondence between X-ray average absorptivities and X-ray photographing parameters according to an X-ray photographing parameter corresponding to a case in which an average gray scale of historical X-ray images of a body part meeting the X-ray average absorptivity at the same photographing angle meets a first preset gray scale range and/or a contrast between two same organs meets a first preset contrast range. In this way, it is conducive to improving the uniformity of the panoramic X-ray image.

In yet another exemplary implementation of the medical X-ray imaging device, edges of photographed regions corresponding to adjacent segmented regions overlap with each other. The parameter generation module can, for each of the segmented regions, further generate the X-ray photographing parameter for the segmented region according to an X-ray photographing parameter corresponding to a case in which an average gray scale of historical X-ray images of a body part meeting an X-ray average absorptivity that is of a body part corresponding to an overlapped part of the photographed regions corresponding to the segmented region at the same photographing angle meets a second preset gray scale range and/or a contrast between two same organs meets a second preset contrast range. In this way, it is conducive to reducing jumps of the gray scale and/or the contrast of the overlapped part.

### BRIEF DESCRIPTION OF THE DRAWINGS

The following accompanying drawings only exemplarily illustrate and explain the present aspects of the disclosure, but are not intended to limit the scope of the present aspects of the disclosure.

FIG. 1 is a flowchart of an exemplary implementation of a medical X-ray imaging method.

FIG. 2 is used for exemplarily illustrating a position and a size of an imaging target region.

FIG. 3 is used for exemplarily illustrating a position and a size of a segmented region.

FIG. 4 is used to show a scale of standard manikins for males and females.

FIG. 5 is used to show an exemplary implementation of a medical bed.

FIG. 6 shows a correspondence between segmented regions and photographed regions.

FIG. 7 is a structural block diagram of an exemplary implementation of a medical X-ray imaging device.

### REFERENCE NUMERALS

100 medical X-ray imaging device
10 target region setting module
20 segmentation module
30 parameter generation module
40 photographing module
50 splicing module
60 first data generation module
70 visible light image obtaining device
80 medical bed
81 bearing surface
811 bearing region
812 pressure detection portion
90 correspondence generation module
102 imaging target region
103 segmented region
104 photographed region
105 overlapped part

### DETAILED DESCRIPTION

To have a clearer understanding of the technical features, the objectives, and the effects of the present disclosure, specific implementations of the present disclosure are now illustrated with reference to the accompanying drawings, and the same reference numerals in the accompanying drawings represent components with the same structure or similar structures but with the same function.

In this specification, “exemplary” indicates “serving as an example, a case, or description”, and any illustration or implementation described as “schematic” in this specification should not be interpreted as a more preferred or more advantageous technical solution.

In this specification, terms such as “first” and “second” do not represent the degree of importance, order, or the like, but are only used to represent a difference between each other to facilitate the description of the specification.

For brevity of the accompanying drawings, only parts related to the present disclosure are exemplarily shown in the accompanying drawings, and do not represent an actual structure as a product.

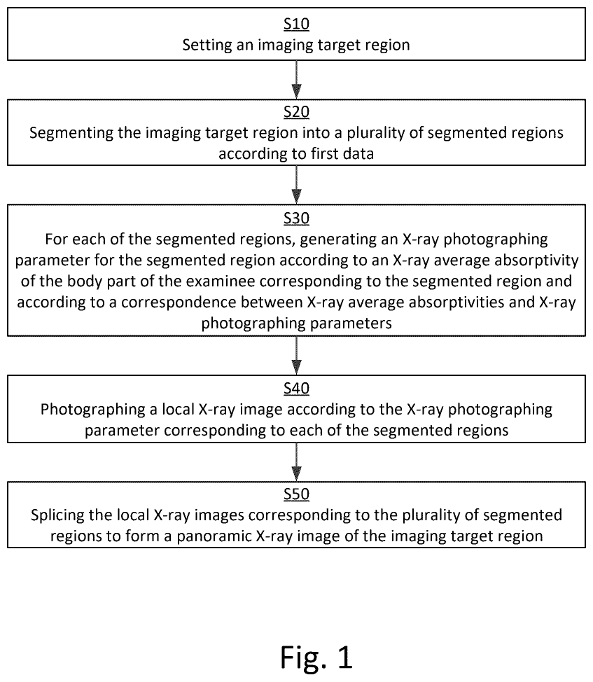

FIG. 1 is a flowchart of an exemplary implementation of a medical X-ray imaging method. The method is, for example, used in X-ray plane scanning. As shown in FIG. 1, the medical X-ray imaging method includes the following steps S10 to S50.

S10: Set an imaging target region.

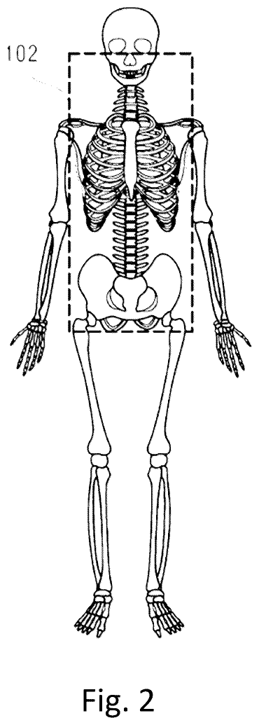

For example, during the making of a panoramic X-ray image of a complete spine, as shown in FIG. 2, the imaging target region 102 may be set as a rectangular region in FIG. 2. A schematic diagram of a human skeleton in FIG. 2 is only to facilitate the illustration of the position and size of the imaging target region, and is not a picture shown during actual use. The imaging target region may be set as required, or, for example, the imaging target region may include a lower half, including a neck, and an upper half, including a chest.

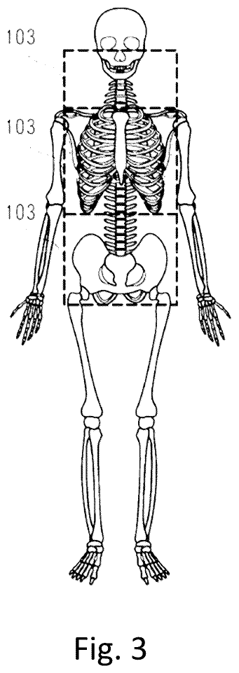

S20: Segment the imaging target region into a plurality of segmented regions according to first data, where the first data is related to a distribution of X-ray absorptivities of body parts of an examinee corresponding to the imaging target region. Specifically, the first data is the distribution of the X-ray absorptivities of the body parts of the examinee corresponding to the imaging target region, and for each of the segmented regions, a difference between a maximum value and a minimum value of an X-ray absorptivity of a body part of the examinee corresponding to the segmented region is not beyond a first preset difference range. For example, the number of segmented regions may be reduced by optimization to reduce the number of exposures. For example, the imaging target region 102 shown in FIG. 2 is segmented into three rectangular segmented regions 103 shown in FIG. 3, and the three segmented regions 103 are arranged in a direction parallel to the long sides of the imaging target region 102. The X-ray absorptivity herein is an X-ray absorptivity in a direction parallel to an X-ray center line. An X-ray that is emitted from a spherical target surface and is perpendicular to a window center is referred to as an X-ray center line. The X-ray center line is representative of a projection direction. The X-ray absorptivity is also related to a density of the human body, in addition to a thickness of the human body in the direction parallel to the X-ray center line. The human body has approximately the same density of tissues other than the bones. A lung is similar to other organs in terms of the density of constituent tissues, but the lung is an inflated tissue when the lung is alive. The X-ray absorptivity of gas is significantly different from that of blood or muscle.

The distribution of the X-ray absorptivities of the body parts of the examinee corresponding to the imaging target region is represented, for example, by using X-ray absorptivities of the examinee along cross sections parallel to the X-ray center line, or by using X-ray absorptivities of body parts of the examinee corresponding to pixel points of the imaging target region. In an exemplary implementation, the distribution of the X-ray absorptivities of the body parts of the examinee corresponding to the imaging target region may further be represented as positions and sizes of local parts of the examinee, where the local parts of the examinee do not overlap with each other, and distinction of the local parts is related to the distribution of the X-ray absorptivities of the examinee. Compared with the X-ray absorptivities of other local parts, the X-ray absorptivities in the same local part are closer, and meet that the difference between the maximum value and the minimum value of the X-ray absorptivity of the body part is not beyond a first preset difference range. In an exemplary implementation, the human body is divided into five local parts, head and neck, chest, abdomen, upper limbs, and lower limbs, but is not limited thereto. In other exemplary implementations, the human body may alternatively be divided into six local parts, for example, head, neck, chest, abdomen, upper limbs, and lower limbs. In this case, in step S20, each segmented region covers only a part or all of one local part of the examinee. As shown in FIG. 3, the upper segmented region 103 covers the head and neck of the examinee, the middle segmented region 103 covers the chest of the examinee, and the lower segmented region 103 covers the abdomen of the examinee.

In the above aspect, according to whether the difference between the maximum value and the minimum value of the X-ray absorptivity of the body part of the examinee corresponding to the segmented region is not beyond a first preset difference range, the first data may be obtained, for example, according to the positions and the sizes of the body parts of the examinee. A correspondence between the first data and the positions and the sizes of the body parts of the examinee is obtained, for example, by machine learning. A division of the body parts is obtained according to the differences between maximum values and minimum values of the X-ray absorptivities corresponding to the positions and the sizes of the body parts in a sample is not beyond a first preset difference range.

In an exemplary implementation, step S20 further includes: obtaining the first data according to an optical image of the examinee. The optical image is a visible light image or a pre-scanned X-ray image. An X-ray pre-scan is a scan before an X-ray imaging scan at a dose less than a dose used during the X-ray imaging scan. The optical image is a 2D image or a 3D image. A posture of the optical image of the examinee needs to be consistent with a posture of an X-ray image.

Specifically, in an exemplary implementation, the optical image is a head image of the examinee. Step S20 includes: obtaining a position and a size of the head of the examinee through measurement according to the head image of the examinee, estimating positions and sizes of body parts of the examinee according to the position and the size of the head of the examinee, and obtaining the first data according to the positions and the sizes of the body parts of the examinee.

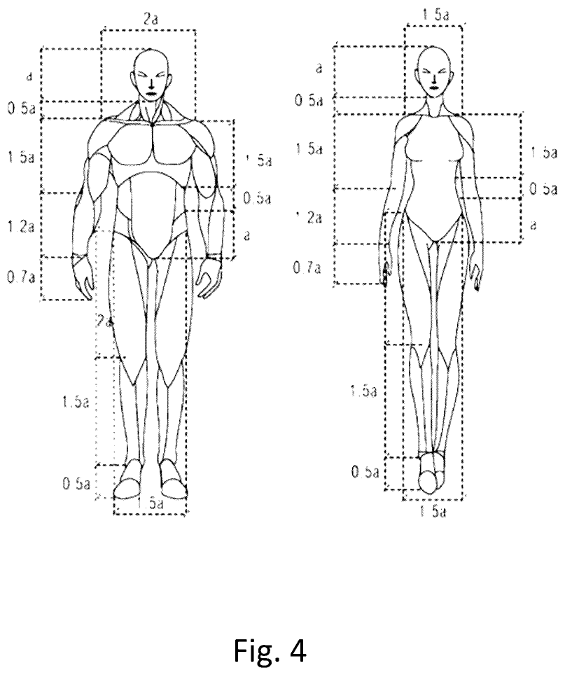

As shown in FIG. 4, the human body is formed by body parts to a scale. If a head size is defined as a length of the body parts can be calculated. This calculation may be an estimation based on a scale of a standard manikin, or may be a calculation based on a body scale obtained from previous measurements of the examinee. It can be understood that the latter is more accurate. FIG. 4 shows a scale of standard manikins for males and females.

In other exemplary implementations, the optical image may alternatively be a whole body image of the examinee. Step S20 includes: obtaining positions and sizes of body parts of the examinee according to the whole body image of the examinee, and obtaining the first data according to the positions and the sizes of the body parts of the examinee.

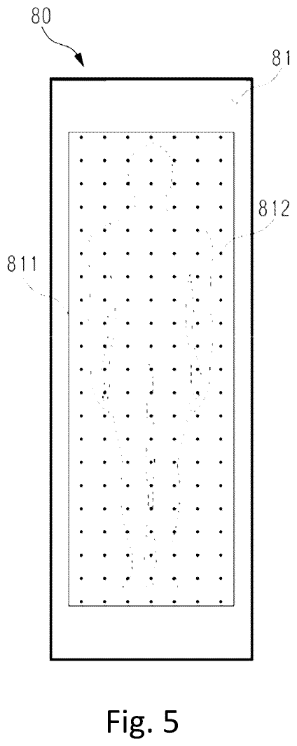

In addition to obtaining the first data according to the optical image of the examinee, the first data may alternatively be obtained in other manners. For example, in another exemplary implementation, as shown in FIG. 5, a bearing surface 81 of a medical bed 80 has a bearing region 811. During imaging, the examinee is borne on the bearing region 811 of the bearing surface 81 of the medical bed 80. A plurality of pressure detection portions 811 are arranged in the bearing region 812. The plurality of pressure detection portions 812 may be uniformly arranged or non-uniformly arranged. The pressure detection portions 812 are, for example, point-shaped or may be linear in other exemplary implementations. The medical bed 80 can detect a pressure of the examinee on the medical bed in a gravity direction at each of the pressure detection portions 812. Step S20 includes: estimating positions and sizes of the body parts of the examinee according to the pressure of the examinee on the medical bed in the gravity direction at the pressure detection portions, and obtaining the first data according to the positions and the sizes of the body parts of the examinee. A correspondence between pressures of the examinee on the medical bed in a gravity direction at the pressure detection portions and the positions and the sizes of the body parts of the examinee may be obtained, for example, by machine learning.

For another example, in other exemplary implementations, step S20 includes: estimating positions and sizes of body parts of the examinee according to a height, a weight, and a body fat ratio of the examinee, and obtaining the first data according to the positions and the sizes of the body parts of the examinee. A correspondence between the height, the weight, and the body fat ratio of the examinee and the positions and the sizes of the body parts of the examinee may be obtained, for example, by machine learning.

S30: generating an X-ray photographing parameter for each segmented region according to the first data. Specifically, for each of the segmented regions, an X-ray photographing parameter for the segmented region is generated according to an X-ray average absorptivity of the body part of the examinee corresponding to the segmented region and according to a correspondence between X-ray average absorptivities and X-ray photographing parameters. The X-ray photographing parameters include, for example, a tube voltage, a tube current, an exposure time, and a photographed region.

Specifically, in the correspondence between the X-ray average absorptivities and the X-ray photographing parameters, for example, an X-ray photographing parameter corresponding to a case in which an average gray scale of historical X-ray images of a body part meeting the X-ray average absorptivity at the same photographing angle meets a first preset gray scale range and/or a contrast between two same organs meets a first preset contrast range is used as an X-ray photographing parameter corresponding to the X-ray average absorptivity. The historical X-ray image may be a historical X-ray image of the examinee or a historical X-ray image of another person. For the same panoramic X-ray image, values of the first preset gray scale range and the first preset contrast range corresponding to the X-ray average absorptivity need to be the same. It may be understood that smaller value ranges of the first preset gray scale range and the first preset contrast range are more conducive to improving the uniformity of the panoramic X-ray image. The correspondence between the X-ray average absorptivities and the X-ray photographing parameters may be obtained, for example, by machine learning, or certainly, may be obtained by the user based on experience.

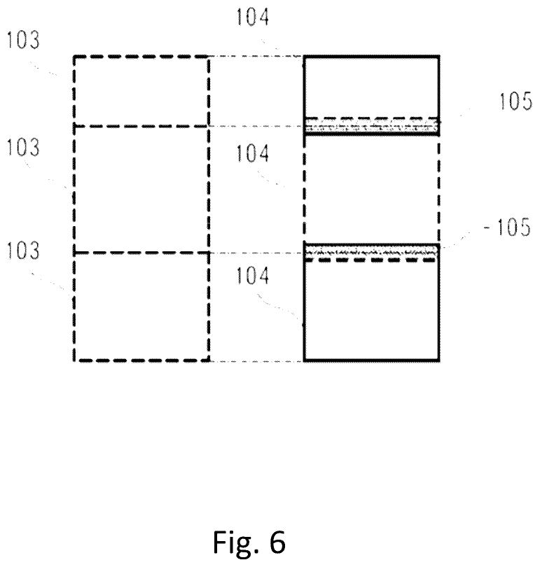

FIG. 6 shows three photographed regions 104 corresponding to the three segmented regions 103 in FIG. 3. A photographed region is an imaging region corresponding to an exposure during medical X-ray imaging. For ease of understanding, adjacent photographed regions 104 in FIG. 6 are drawn using different lines. Each photographed region 104 covers a corresponding segmented region 103, edges of the photographed regions 104 corresponding to adjacent segmented regions 103 overlap with each other, and overlapped parts 105 are shown in FIG. 6 by dotted filling.

Further, in an exemplary implementation, in step S30, for each of the segmented regions, an X-ray photographing parameter that is in X-ray photographing parameters corresponding to a case in which an average gray scale of historical X-ray images of a body part meeting an X-ray average absorptivity that is of a body part corresponding to an overlapped part of the photographed regions corresponding to the segmented region at the same photographing angle meets a second preset gray scale range and/or a contrast between two same organs meets a second preset contrast range and that meets the “correspondence between the X-ray average absorptivities and the X-ray photographing parameters” is used as the X-ray photographing parameter for the segmented region. The historical X-ray image may be a historical X-ray image of the examinee or a historical X-ray image of another person. In this way, it is conducive to reducing jumps of the gray scale and/or the contrast of the overlapped part. It may be understood that smaller value ranges of the second preset gray scale range and the second preset contrast range are more conducive to improving the uniformity of the panoramic X-ray image.

However, this is not limited thereto, and in an exemplary implementation in which the distribution of the X-ray absorptivities of the body part of the examinee corresponding to the imaging target region is represented as the positions and the sizes of the local parts of the examinee, step S30, for example, specifically includes: for each of the segmented regions, generating an X-ray photographing parameter for the segmented region according to the local part of the examinee corresponding to the segmented region and according to a correspondence between the local parts and X-ray photographing parameters.

Specifically, in the correspondence between the local parts and the X-ray photographing parameters, for example, an X-ray photographing parameter corresponding to a case in which an average gray scale of historical X-ray images of the local parts at the same photographing angle meets a first preset gray scale range and/or a contrast between two same organs meets a first preset contrast range is used as an X-ray photographing parameter corresponding to the local part. The contrast between the two same organs represents a gray scale difference between the two same organs. The same organs may be selected from adjacent organs having a large gray scale difference, or one organ may be selected as an organ of interest, and the other may be selected as an organ in the same shot as the organ of interest and have a large gray scale difference. The historical X-ray image may be a historical X-ray image of the examinee or a historical X-ray image of another person. For the same panoramic X-ray image, values of the first preset gray scale range and the first preset contrast range corresponding to each local part need to be the same. It may be understood that smaller value ranges of the first preset gray scale range and the first preset contrast range are more conducive to improving the uniformity of the panoramic X-ray image. The correspondence between the local parts and the X-ray photographing parameters may be obtained, for example, by machine learning, or certainly, may be obtained by the user based on experience. When the average gray scale and the contrast between the two same organs are considered, trade-offs may alternatively be made on the two indicators by considering the two indicators comprehensively. One consideration manner is that, assuming that the gray scale is G and the contrast is C, a range of value U is considered to be within a first preset uniformity range, where U=a*G+b*C, a and b as weight coefficients may be obtained according to experience, and U may be regarded as an indicator of uniformity.

Further, in an exemplary implementation, in step S30, for each of the segmented regions 103, the X-ray photographing parameter for the segmented region is further generated according to an X-ray photographing parameter corresponding to a case in which an average gray scale of historical X-ray images of a body part corresponding to an overlapped part 105 of the photographed regions 104 corresponding to the segmented region 103 meets a second preset gray scale range and/or a contrast between two same organs meets a second preset contrast range. The historical X-ray image may be a historical X-ray image of the examinee or a historical X-ray image of another person. For example, if the body part corresponding to the overlapped part is a body part of a height range in which an n^{th} lumbar vertebra is located, an X-ray photographing parameter that is in X-ray photographing parameters corresponding to a case in which an average gray scale of historical X-ray images of a body part of the height range corresponding to the n^{th} lumbar vertebra of historical X-ray images of the examinee or another person meets a second preset gray scale range and/or a contrast between two same organs meets a second preset contrast range and that meets the “correspondence between the local parts and the X-ray photographing parameters” is used as the X-ray photographing parameter for the segmented region. In this way, it is conducive to reducing jumps of the gray scale and/or the contrast of the overlapped part. It may be understood that smaller value ranges of the second preset gray scale range and the second preset contrast range are more conducive to improving the uniformity of the panoramic X-ray image.

S40: Photograph a local X-ray image according to the X-ray photographing parameter corresponding to each of the segmented regions, where a range of each local X-ray image corresponds to a photographed region.

S50: Splice the local X-ray images corresponding to the plurality of segmented regions to form a panoramic X-ray image of the imaging target region. Image fusion of the overlapped parts is processed according to the method in the related art, and details are not described herein.

In the medical X-ray imaging method in this exemplary implementation, the imaging target region is segmented into the plurality of segmented regions according to the distribution of the X-ray absorptivities of the body parts corresponding to the imaging target region of the examinee, and the X-ray photographing parameter for each of the segmented regions is generated according to the X-ray average absorptivity of the body part corresponding to the segmented region of the examinee and the correspondence between the X-ray average absorptivities and the X-ray photographing parameters. Since the X-ray absorptivities in the body parts corresponding to the same segmented region are close, a consistent exposure degree of parts in the local X-ray images can be achieved, thereby improving the uniformity of the panoramic X-ray image.

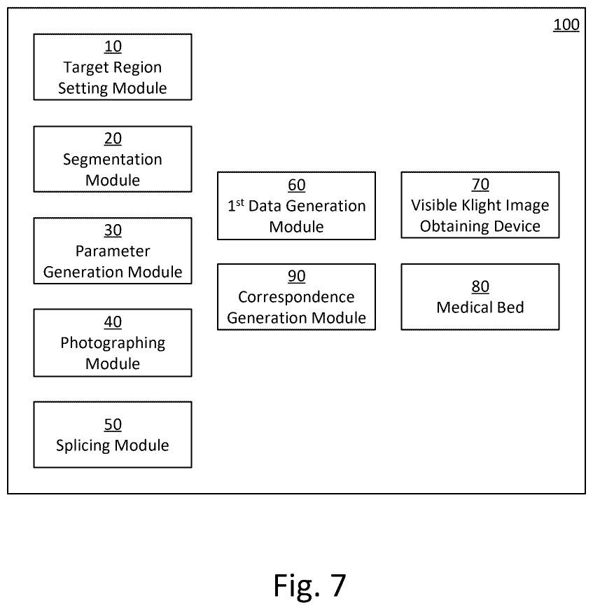

The present disclosure further provides a medical X-ray imaging device. FIG. 7 is a structural block diagram of an exemplary implementation of a medical X-ray imaging device. As shown in FIG. 7, the medical X-ray imaging device 100 includes a target region setting module 10, a segmentation module 20, a parameter generation module 30, a photographing module 40, and a splicing module 50.

The target region setting module 10 can set an imaging target region according to data input by a user. The segmentation module 20 can segment the imaging target region into a plurality of segmented regions according to the first data. The first data is a distribution of X-ray absorptivities of body parts of an examinee corresponding to the imaging target region. The segmentation module 20 can, for each of the segmented regions, maintain a difference between a maximum value and a minimum value of an X-ray absorptivity of a body part of the examinee corresponding to the segmented region, not beyond a first preset difference range. For example, the number of segmented regions may be reduced by optimization to reduce the number of exposures. The X-ray absorptivity herein is an X-ray absorptivity in a direction parallel to an X-ray center line.

The distribution of the X-ray absorptivities of the body parts of the examinee corresponding to the imaging target region is represented, for example, by using X-ray absorptivities of the examinee along cross sections parallel to the X-ray center line, or by using X-ray absorptivities of body parts of the examinee corresponding to pixel points of the imaging target region. In an exemplary implementation, the distribution of the X-ray absorptivities of the body parts of the examinee corresponding to the imaging target region may further be represented as positions and sizes of local parts of the examinee, where the local parts of the examinee do not overlap with each other, and distinction of the local parts is related to the distribution of the X-ray absorptivities of the examinee. In this exemplary implementation, the human body is divided into five local parts: head and neck, chest, abdomen, upper limbs, and lower limbs, but is not limited thereto. In other exemplary implementations, the human body may alternatively be divided into six local parts, for example, head, neck, chest, abdomen, upper limbs, and lower limbs. Compared with the X-ray absorptivities of other local parts, the X-ray absorptivities in the same local part are closer. The segmentation module 20 can enable each of the segmented regions to cover only a part or all of one local part of the examinee.

The first data is, for example, obtained according to positions and sizes of body parts of the examinee. A correspondence between the first data and the positions and the sizes of the body parts of the examinee is obtained, for example, by machine learning.

In an exemplary implementation, the medical X-ray imaging device 100 further includes a first data generation module 60. The first data generation module 60 can obtain the first data according to an optical image of the examinee. The optical image is a visible light image or a pre-scanned X-ray image. In a case in which the optical image is a visible light image, the medical X-ray imaging device 100 further includes a visible light image obtaining device 70. The visible light image obtaining device 70 is configured to obtain the visible light image of the examinee. The medical X-ray imaging device, for example, obtains a pre-scanned X-ray image of the examinee through the photographing module 40. The optical image is a 2D image or a 3D image. A posture of the optical image of the examinee needs to be consistent with a posture of an X-ray image.

Specifically, in an exemplary implementation, the optical image is a head image of the examinee. The first data generation module 60 can obtain a position and a size of the head of the examinee through measurement according to the head image of the examinee, estimate positions and sizes of body parts of the examinee according to the position and the size of the head of the examinee, and obtain the first data according to the positions and the sizes of the body parts of the examinee.

In other exemplary implementations, the optical image is a whole body image of the examinee. The first data generation module 60 can obtain positions and sizes of body parts of the examinee according to the whole body image of the examinee, and obtain the first data according to the positions and the sizes of the body parts of the examinee.

In addition to obtaining the first data according to the optical image of the examinee, the first data may alternatively be obtained in other manners. For example, in another exemplary implementation, as shown in FIG. 5, the medical X-ray imaging device 100 further includes a medical bed 80, and a bearing surface 81 of the medical bed 80 has a bearing region 811. A plurality of pressure detection portions 811 are arranged in the bearing region 812. The plurality of pressure detection portions 812 may be uniformly arranged or non-uniformly arranged. The pressure detection portions 812 are, for example, point-shaped or may be linear in other exemplary implementations. The medical bed 80 can detect a pressure of the examinee on the medical bed 80 in a gravity direction at each of the pressure detection portions 812. The first data generation module 60 can estimate positions and sizes of body parts of the examinee according to the pressure of the examinee on the medical bed 80 in the gravity direction at the pressure detection portions 812, and obtain the first data according to the positions and the sizes of the body parts of the examinee. A correspondence between pressures of the examinee on the medical bed in a gravity direction at the pressure detection portions and the positions and the sizes of the body parts of the examinee may be obtained, for example, by machine learning.

For another example, in an exemplary implementation, the first data generation module 60 can estimate positions and sizes of body parts of the examinee according to a height, a weight, and a body fat ratio of the examinee, and obtain the first data according to the positions and the sizes of the body parts of the examinee. A correspondence between the height, the weight, and the body fat ratio of the examinee and the positions and the sizes of the body parts of the examinee may be obtained, for example, by machine learning.

The parameter generation module 30 can, for each of the segmented regions, generate an X-ray photographing parameter for the segmented region according to an X-ray average absorptivity of the body part of the examinee corresponding to the segmented region and according to a correspondence between X-ray average absorptivities and X-ray photographing parameters. The X-ray photographing parameters include, for example, a tube voltage, a tube current, an exposure time, and a photographed region.

Specifically, in an exemplary implementation, the medical X-ray imaging device 100 further includes a correspondence generation module 90. The correspondence generation module 90 can use an X-ray photographing parameter corresponding to a case in which an average gray scale of historical X-ray images of a body part meeting the X-ray average absorptivity at the same photographing angle meets a first preset gray scale range and/or a contrast between two same organs meets a first preset contrast range as the X-ray photographing parameter corresponding to the X-ray average absorptivity. The historical X-ray image may be a historical X-ray image of the examinee or a historical X-ray image of another person. For the same panoramic X-ray image, values of the first preset gray scale range and the first preset contrast range corresponding to the X-ray average absorptivity need to be the same. It may be understood that smaller value ranges of the first preset gray scale range and the first preset contrast range are more conducive to improving the uniformity of the panoramic X-ray image. The correspondence between the X-ray average absorptivities and the X-ray photographing parameters may be obtained, for example, by machine learning, or certainly, may be obtained by the user based on experience.

Further, in an exemplary implementation, the parameter generation module 30 can, for each of the segmented regions, use an X-ray photographing parameter that is in X-ray photographing parameters corresponding to a case in which an average gray scale of historical X-ray images of a body part meeting an X-ray average absorptivity that is of a body part corresponding to an overlapped part of the photographed regions corresponding to the segmented region at the same photographing angle meets a second preset gray scale range and/or a contrast between two same organs meets a second preset contrast range and that meets the “correspondence between the X-ray average absorptivities and the X-ray photographing parameters” as the X-ray photographing parameter for the segmented region. The historical X-ray image may be a historical X-ray image of the examinee or a historical X-ray image of another person. In this way, it is conducive to reducing jumps of the gray scale and/or the contrast of the overlapped part. It may be understood that smaller value ranges of the second preset gray scale range and the second preset contrast range are more conducive to improving the uniformity of the panoramic X-ray image.

However, this is not limited thereto. In an exemplary implementation in which the distribution of the X-ray absorptivities of the body parts of the examinee corresponding to the imaging target region is represented as the positions and the sizes of the local parts of the examinee, the parameter generation module 30 can, for each of the segmented regions, generate an X-ray photographing parameter for the segmented region according to the local part of the examinee corresponding to the segmented region and according to a correspondence between the local parts and X-ray photographing parameters.

Specifically, the medical X-ray imaging device 100 further includes a correspondence generation module 90. The correspondence generation module 90 can use an X-ray photographing parameter corresponding to a case in which an average gray scale of historical X-ray images of the local parts at the same photographing angle meets a first preset gray scale range and/or a contrast between two same organs meets a first preset contrast range as an X-ray photographing parameter corresponding to the local part. The historical X-ray image may be a historical X-ray image of the examinee or a historical X-ray image of another person. For the same panoramic X-ray image, values of the first preset gray scale range and the first preset contrast range corresponding to each local part need to be the same. It may be understood that smaller value ranges of the first preset gray scale range and the first preset contrast range are more conducive to improving the uniformity of the panoramic X-ray image. The correspondence between the local parts and the X-ray photographing parameters may be obtained, for example, by machine learning, or certainly, may be obtained by the user based on experience.

Further, in an exemplary implementation, the parameter generation module 30 can, for each of the segmented regions, further generate the X-ray photographing parameter for the segmented region according to an X-ray photographing parameter corresponding to a case in which an average gray scale of historical X-ray images of a body part corresponding to an overlapped part of the photographed regions corresponding to the segmented region meets a second preset gray scale range and/or a contrast between two same organs meets a second preset contrast range. The historical X-ray image may be a historical X-ray image of the examinee or a historical X-ray image of another person. For example, if the body part corresponding to the overlapped part is a body part of a height range in which an n^{th} lumbar vertebra is located, an X-ray photographing parameter that is in X-ray photographing parameters corresponding to a case in which an average gray scale of historical X-ray images of a body part of the height range corresponding to the n^{th} lumbar vertebra of historical X-ray images of the examinee or another person meets a second preset gray scale range and/or a contrast between two same organs meets a second preset contrast range and that meets the “correspondence between the local parts and the X-ray photographing parameters” is used as the X-ray photographing parameter for the segmented region. In this way, it is conducive to reducing jumps of the gray scale and/or the contrast of the overlapped part. It may be understood that smaller value ranges of the second preset gray scale range and the second preset contrast range are more conducive to improving the uniformity of the panoramic X-ray image.

The photographing module 40 can photograph a local X-ray image according to the X-ray photographing parameter corresponding to each of the segmented regions. The splicing module 50 can splice the local X-ray images corresponding to the plurality of segmented regions to form a panoramic X-ray image of the imaging target region.

In the medical X-ray imaging device in this exemplary implementation, the imaging target region is segmented into the plurality of segmented regions according to the distribution of the X-ray absorptivities of the body parts corresponding to the imaging target region of the examinee, and the X-ray photographing parameter for each of the segmented regions is generated according to the X-ray average absorptivity of the body part corresponding to the segmented region of the examinee and the correspondence between the X-ray average absorptivities and the X-ray photographing parameters. Since the X-ray absorptivities in the body parts corresponding to the same segmented region are close, a consistent exposure degree of parts in the local X-ray images can be achieved, thereby improving the uniformity of the panoramic X-ray image.

It should be understood that, although this specification is described according to each aspect, each aspect may not include only one independent technical solution. The description manner of this specification is merely for clarity. This specification should be considered as a whole by a person skilled in the art, and the technical solution in each aspect may also be properly combined to form other implementations that can be understood by a person skilled in the art.

The series of detailed descriptions set forth above are merely specific illustrations of possible aspects of the present disclosure and are not intended to limit the scope of the aspects of the present disclosure. Equivalent implementations or variations, such as combinations, divisions, or repetitions of features, which do not depart from the spirit of the aspects of the present disclosure, are intended to be included within the scope of the aspects of the present disclosure.

1-28. (canceled)

29. A medical X-ray imaging method, comprising:
S10: setting an imaging target region;
S20: segmenting the imaging target region into a plurality of segmented regions according to first data, wherein the first data is a distribution of X-ray absorptivities of body parts of an examinee corresponding to the imaging target region, and for each of the segmented regions, a difference between a maximum value and a minimum value of an X-ray absorptivity of a body part of the examinee corresponding to the segmented region is not beyond a first preset difference range;
S30: for each of the segmented regions, generating an X-ray photographing parameter for the segmented region according to an X-ray average absorptivity of the body part of the examinee corresponding to the segmented region and according to a correspondence between X-ray average absorptivities and X-ray photographing parameters;
S40: photographing a local X-ray image according to the X-ray photographing parameter corresponding to each of the segmented regions; and
S50: splicing the local X-ray images corresponding to the plurality of segmented regions to form a panoramic X-ray image of the imaging target region.

30. The medical X-ray imaging method according to claim 29, wherein the distribution of the X-ray absorptivities of the body parts of the examinee corresponding to the imaging target region is represented as positions and sizes of local parts of the examinee, and wherein the local parts of the examinee do not overlap with each other, distinction of the local parts is related to the distribution of the X-ray absorptivities of the examinee, and in the step S20, each of the segmented regions covers only a part or all of one local part of the examinee.

31. The medical X-ray imaging method according to claim 30, wherein one of the local parts is a head, neck, head and neck, chest, abdomen, upper limb, or lower limb.

32. The medical X-ray imaging method according to claim 29, wherein the step S20 comprises: obtaining the first data according to an optical image of the examinee, wherein the optical image is a visible light image or a pre-scanned X-ray image, and the optical image is a 2D image or a 3D image.

33. The medical X-ray imaging method according to claim 32, wherein the optical image is a head image of the examinee, and the step S20 comprises: obtaining a position and a size of the head of the examinee through measurement according to the head image of the examinee, estimating positions and sizes of body parts of the examinee according to the position and the size of the head of the examinee, and obtaining the first data according to the positions and the sizes of the body parts of the examinee.

34. The medical X-ray imaging method according to claim 32, wherein the optical image is a whole body image of the examinee, and the step S20 comprises: obtaining positions and sizes of body parts of the examinee according to the whole body image of the examinee, and obtaining the first data according to the positions and the sizes of the body parts of the examinee.

35. The medical X-ray imaging method according to claim 29, wherein the examinee is borne on a bearing surface of a medical bed, the bearing surface has a bearing region, a plurality of pressure detection portions are arranged in the bearing region, the medical bed is capable of detecting a pressure of the examinee on the medical bed in a gravity direction at each of the pressure detection portions, and the step S20 comprises: estimating positions and sizes of body parts of the examinee according to the pressure of the examinee on the medical bed in the gravity direction at the pressure detection portions, and obtaining the first data according to the positions and the sizes of the body parts of the examinee.

36. The medical X-ray imaging method according to claim 29, wherein the step S20 comprises: estimating positions and sizes of body parts of the examinee according to a height, a weight, and a body fat ratio of the examinee, and obtaining the first data according to the positions and the sizes of the body parts of the examinee.

37. The medical X-ray imaging method according to claim 30, wherein the step S30 comprises: for each of the segmented regions, generating an X-ray photographing parameter for the segmented region according to the local part of the examinee corresponding to the segmented region and according to a correspondence between the local parts and X-ray photographing parameters.

38. The medical X-ray imaging method according to claim 37, wherein the correspondence between the local parts and X-ray photographing parameters is determined according to an X-ray photographing parameter corresponding to a case in which an average gray scale of historical X-ray images of the local parts at the same photographing angle meets a first preset gray scale range and/or a contrast between two same organs meets a first preset contrast range.

39. The medical X-ray imaging method according to claim 38, wherein edges of photographed regions corresponding to adjacent segmented regions overlap with each other, and in the step S30, for each of the segmented regions, the X-ray photographing parameter for the segmented region is further generated according to an X-ray photographing parameter corresponding to a case in which an average gray scale of historical X-ray images of a body part corresponding to an overlapped part of the photographed regions corresponding to the segmented region meets a second preset gray scale range and/or a contrast between two same organs meets a second preset contrast range.

40. The medical X-ray imaging method according to claim 29, wherein the correspondence between X-ray average absorptivities and X-ray photographing parameters is determined according to an X-ray photographing parameter corresponding to a case in which an average gray scale of historical X-ray images of a body part meeting the X-ray average absorptivity at the same photographing angle meets a first preset gray scale range and/or a contrast between two same organs meets a first preset contrast range.

41. The medical X-ray imaging method according to claim 40, wherein edges of photographed regions corresponding to adjacent segmented regions overlap with each other, and in the step S30, for each of the segmented regions, the X-ray photographing parameter for the segmented region is further generated according to an X-ray photographing parameter corresponding to a case in which an average gray scale of historical X-ray images of a body part meeting an X-ray average absorptivity that is of a body part corresponding to an overlapped part of the photographed regions corresponding to the segmented region at the same photographing angle meets a second preset gray scale range and/or a contrast between two same organs meets a second preset contrast range.

42. The medical X-ray imaging method according to claim 29, wherein the X-ray photographing parameters comprise a tube voltage, a tube current, an exposure time, and a photographed region.

43. The medical X-ray imaging method according to claim 29, wherein the imaging target region is a rectangle, and the plurality of segmented regions are arranged in a direction parallel to a set of opposite edges of the rectangle.

44. A medical X-ray imaging device, comprising:
a target region setting module configured to set an imaging target region according to data input by a user;
a segmentation module configured to segment the imaging target region into a plurality of segmented regions according to first data, wherein the first data is a distribution of X-ray absorptivities of body parts of an examinee corresponding to the imaging target region, and the segmentation module is configured to: for each of the segmented regions, maintain a difference between a maximum value and a minimum value of an X-ray absorptivity of a body part of the examinee corresponding to the segmented region not beyond a first preset difference range;
a parameter generation module configured to: for each of the segmented regions, generate an X-ray photographing parameter for the segmented region according to an X-ray average absorptivity of the body part of the examinee corresponding to the segmented region and according to a correspondence between X-ray average absorptivities and X-ray photographing parameters;
a photographing module configured to photograph a local X-ray image according to the X-ray photographing parameter corresponding to each of the segmented regions; and
a splicing module configured to splice the local X-ray images corresponding to the plurality of segmented regions to form a panoramic X-ray image of the imaging target region.

45. The medical X-ray imaging device according to claim 44, wherein the distribution of the X-ray absorptivities of the body parts of the examinee corresponding to the imaging target region is represented as positions and sizes of local parts of the examinee, wherein the local parts of the examinee do not overlap with each other, distinction of the local parts is related to the distribution of the X-ray absorptivities of the examinee, and the segmentation module is configured to enable each of the segmented regions to cover only a part or all of one local part of the examinee.

46. The medical X-ray imaging device according to claim 45, wherein one of the local parts is a head, neck, head and neck, chest, abdomen, upper limb, or lower limb.

47. The medical X-ray imaging device according to claim 44, further comprising a first data generation module configured to obtain the first data according to an optical image of the examinee, wherein the optical image is a visible light image or a pre-scanned X-ray image, and in a case in which the optical image is a visible light image, the medical X-ray imaging device further comprises a visible light image obtaining device configured to obtain the visible light image of the examinee, and the optical image is a 2D image or a 3D image.

48. The medical X-ray imaging device according to claim 47, wherein the optical image is a head image of the examinee, and the first data generation module is configured to obtain a position and a size of the head of the examinee through measurement according to the head image of the examinee, estimate positions and sizes of body parts of the examinee according to the position and the size of the head of the examinee, and obtain the first data according to the positions and the sizes of the body parts of the examinee.

49. The medical X-ray imaging device according to claim 47, wherein the optical image is a whole body image of the examinee, and the first data generation module is configured to obtain positions and sizes of body parts of the examinee according to the whole body image of the examinee, and obtain the first data according to the positions and the sizes of the body parts of the examinee.

50. The medical X-ray imaging device according to claim 44, further comprising a first data generation module, wherein the examinee is borne on a bearing surface of a medical bed of the medical X-ray imaging device, the bearing surface has a bearing region, a plurality of pressure detection portions are arranged in the bearing region, the medical bed is configured to detect a pressure of the examinee on the medical bed in a gravity direction at each of the pressure detection portions, and the first data generation module is configured to estimate positions and sizes of body parts of the examinee according to the pressure of the examinee on the medical bed in the gravity direction at the pressure detection portions, and obtain the first data according to the positions and the sizes of the body parts of the examinee.

51. The medical X-ray imaging device according to claim 44, further comprising a first data generation module configured to estimate positions and sizes of body parts of the examinee according to a height, a weight, and a body fat ratio of the examinee, and obtain the first data according to the positions and the sizes of the body parts of the examinee.

52. The medical X-ray imaging device according to claim 45, wherein the parameter generation module is configured to: for each of the segmented regions, generate an X-ray photographing parameter for the segmented region according to the local part of the examinee corresponding to the segmented region and according to a correspondence between the local parts and X-ray photographing parameters.

53. The medical X-ray imaging device according to claim 52, further comprising a correspondence generation module configured to determine the correspondence between the local parts and X-ray photographing parameters according to an X-ray photographing parameter corresponding to a case in which an average gray scale of historical X-ray images of the local parts at the same photographing angle meets a first preset gray scale range and/or a contrast between two same organs meets a first preset contrast range.

54. The medical X-ray imaging device according to claim 53, wherein edges of photographed regions corresponding to adjacent segmented regions overlap with each other, and the parameter generation module is configured to: for each of the segmented regions, further generate the X-ray photographing parameter for the segmented region according to an X-ray photographing parameter corresponding to a case in which an average gray scale of historical X-ray images of a body part corresponding to an overlapped part of the photographed regions corresponding to the segmented region meets a second preset gray scale range and/or a contrast between two same organs meets a second preset contrast range.

55. The medical X-ray imaging device according to claim 44, further comprising a correspondence generation module configured to determine the correspondence between X-ray average absorptivities and X-ray photographing parameters according to an X-ray photographing parameter corresponding to a case in which an average gray scale of historical X-ray images of a body part meeting the X-ray average absorptivity at the same photographing angle meets a first preset gray scale range and/or a contrast between two same organs meets a first preset contrast range.

56. The medical X-ray imaging device according to claim 55, wherein edges of photographed regions corresponding to adjacent segmented regions overlap with each other, and the parameter generation module is configured to: for each of the segmented regions, further generate the X-ray photographing parameter for the segmented region according to an X-ray photographing parameter corresponding to a case in which an average gray scale of historical X-ray images of a body part meeting an X-ray average absorptivity that is of a body part corresponding to an overlapped part of the photographed regions corresponding to the segmented region at the same photographing angle meets a second preset gray scale range and/or a contrast between two same organs meets a second preset contrast range.
